(19) United States
(12) Patent Application Publication
(10) Pub. No.: US 2025/0199333 A1
(43) Pub. Date: Jun. 19, 2025

(54) LENS WITH IMPROVED VISUAL PERFORMANCE

(71) Applicant: Essilor International, Charenton Le Pont (FR)

(72) Inventors: Matthieu GUILLOT, Charenton Le Pont (FR); Eric GACOIN, Charenton Le Pont (FR); David RIO, Charenton Le Pont (FR); Samy HAMLAOUI, Charenton Le Pont (FR); Guillaume GIRAUDET, Charenton Le Pont (FR)

(73) Assignee: Essilor International, Charenton Le Pont (FR)

(21) Appl. No.: 18/846,299

(22) PCT Filed: Apr. 21, 2023
(86) PCT No.: PCT/EP2023/060547

§ 371 Date: Sep. 12, 2024

(30) Foreign Application Priority Data
Apr. 21, 2022 (EP) 22305590.6

(51) Int. Cl. G02C 7/02

(52) U.S. Cl. CPC G02C 7/022

(57) ABSTRACT

It is proposed a lens intended to be worn in front of or on an eye of a wearer and to provide a first refractive power (Rx) for correcting an abnormal refraction of the eye of the wearer; wherein the lens comprises a refractive area configured to provide the first refractive power (Rx) and a functional area configured to provide an optical function; and wherein on a first zone defined on the lens, more than 5% of the surface of said first zone has a local refractive power ranging from the first refractive power (Rx) minus 0.25 diopter and the first refractive power (Rx) plus 0.25 diopter, and more than 25% of the surface of said first zone covers the functional area.

### FIELD OF THE INVENTION

The present invention pertains to the field of ophthalmic lens.

More specifically, the present invention relates to a lens intended to be worn by a wearer comprising a refractive area configured to correct an abnormal refraction of the eye of the wearer and a functional area.

### BACKGROUND OF THE INVENTION

Several solutions exist for suppressing or slowing down progression of an abnormal refraction.

WO2012/034265 discloses a first solution for retarding the progression of myopia by providing a concentric annular multi-zone refractive lens including at least one correcting zone of optical power for correcting refractive error and at least one defocusing zone for projecting at least one non-homogenous defocused image in front of at least a part of a retina to inhibit myopic eye growth, wherein the correcting and defocusing zones are alternated and wherein the lens has a central zone that is a circular first correcting zone.

US2017/0131567 discloses a second solution to suppress progression of myopia by providing a spectacle lens comprising a first correcting area and a plurality of independent island-shaped areas in the vicinity of a center part of the lens configured to focus an image on a position other than a retina of the eye.

WO2019/1666653 disclose a third solution to slow down the progression of an abnormal refraction of the eye by providing a lens comprising a central prescription portion and a plurality of optical elements having a non-spherical optical function.

WO2019/152438 discloses a fourth solution for treating eye-length disorder by providing an ophthalmic lens comprising a scattering region surrounding a clear aperture.

All these solutions comprise a refractive area configured to provide a refractive power (Rx) for correcting an abnormal refraction and a functional area to slow down the abnormal refraction progression. Due to the arrangement of the refractive and functional areas, which are usually alternated, it is difficult to achieve the refractive power (Rx) over the whole refractive area, especially in the vicinity of the functional area. It is also difficult to obtain the refractive power (Rx) over the whole refractive area due to the shape of the surface of the functional area that may be required to obtain the optical function.

This is particularly the case if the functional area comprises optical elements on the surface of the lens. When the surface of the lens is covered by a coating, the coating covering the optical elements has a negative effect on the curvature of the refractive area, for instance in the vicinity of the optical elements. The deposit of the coating will not follow the theoretical discontinuity in power at the junction between the optical elements and the functional area. These defects create a refractive power error—the power of the refractive area will differ from the power prescribed by the eye care practitioner—that reduce the visual performance. Said reduction in the visual performance is independent of the effect of the optical elements on the lens as such, that also reduces the visual performance.

Accordingly, there is a need to quantify the refractive power error and to provide a lens comprising a functional area and a refractive area, wherein the lens preserves the visual performance.

The present invention lies within this context.

### SUMMARY OF THE INVENTION

According to a first aspect of the invention, there is provided a lens intended to be worn in front of or on an eye of a wearer and to provide a first refractive power (Rx) for correcting an abnormal refraction of the eye of the wearer; wherein the lens comprises a refractive area configured to provide the first refractive power (Rx) and a functional area configured to provide an optical function; and wherein on a first zone defined on the lens, more than 5%, for example more than 5%, of the surface of said first zone has a local refractive power ranging from the first refractive power (Rx) minus 0.25 diopter and the first refractive power (Rx) plus 0.25 diopter, and more than 25%, for example more than 30%, of the surface of said first zone covers the functional area and less than 60% of the surface of said first zone covers the functional area.

According to a further aspect of the invention, there is provided a spectacle lens intended to be worn in front of or on an eye of a wearer and to provide a first refractive power (Rx) for correcting an abnormal refraction of the eye of the wearer; wherein
the lens comprises a refractive area configured to provide the first refractive power (Rx) and on at least one surface a functional area configured to provide an optical function; and a coating covering the at least one surface having the functional area,
wherein
   on a first zone having a circular shape defined on the lens, more than 5%, for example more than 5%, for example more than 7%, and less than 39% of the surface of said first zone has a local refractive power ranging from the first refractive power (Rx) minus 0.25 diopter and the first refractive power (Rx) plus 0.25 diopter, and more than 25%, for example more than 30% and less than 60% of the surface of said first zone covers the functional area.

Advantageously, the range of values of the local refractive power ensures that the prescribed refractive power (Rx) and thus the visual performance of the lens will be preserved.

According to other embodiments of the first aspect, the proposed lens may also comprise at least one of the following additional features:
more than 10% of the surface of said first zone has a local refractive power ranging from the first refractive power (Rx) minus 0.25 diopter and the first refractive power (Rx) plus 0.25 diopter; or more than 15% of the surface of said first zone has a local refractive power ranging from the first refractive power (Rx) minus 0.25 diopter and the first refractive power (Rx) plus 0.25 diopter; or more than 20% of the surface of said first zone has a local refractive power ranging from the first refractive power (Rx) minus 0.25 diopter and the first refractive power (Rx) plus 0.25 diopter; or more than 25% of the surface of said first zone has a local refractive power ranging from the first refractive power (Rx) minus 0.25 diopter and the first refractive power (Rx) plus 0.25 diopter; and/or
more than 15% of the surface of said first zone has a local refractive power ranging from the first refractive power (Rx) minus 0.5 diopter and the first refractive power (Rx) plus 0.5 diopter; or more than 20% of the surface of said first zone has a local refractive power ranging from the first refractive power (Rx) minus 0.5 diopter and the first refractive power (Rx) plus 0.5 diopter; or more than 25% of the surface of said first zone has a local refractive power ranging from the first refractive power (Rx) minus 0.5 diopter and the first refractive power (Rx) plus 0.5 diopter; or more than 30% of the surface of said first zone has a local refractive power ranging from the first refractive power (Rx) minus 0.5 diopter and the first refractive power (Rx) plus 0.5 diopter; or more than 35% of the surface of said first zone has a local refractive power ranging from the first refractive power (Rx) minus 0.5 diopter and the first refractive power (Rx) plus 0.5 diopter; and/or
less than 39% of the surface of said first zone has a local refractive power superior or equal to the first refractive power (Rx) plus 0.12 diopter; or less than 37% of the surface of said first zone has a local refractive power superior or equal to the first refractive power (Rx) plus 0.12 diopter; or less than 30% of the surface of said first zone has a local refractive power superior or equal to the first refractive power (Rx) plus 0.12 diopter; and/or
the first zone is a circular zone having a diameter ranging from 6 mm to 10 mm; and/or
the first zone is a circular zone having a diameter greater than or equal to 6 mm and smaller than or equal to 10 mm; and/or
the functional area has an optical function of not focusing an image on the retina of the eye of the wearer or an optical function of scattering incident light; and/or
the functional area is configured to provide an optical function of not focusing an image on the retina of the eye of the wearer or an optical function of scattering incident light; and/or
the functional area has an optical function of not focusing an image on the retina of the eye of the wearer or an optical function of scattering incident light so as to suppress or slow down the progression of the abnormal refraction of the eye of the wearer; and/or
the functional area is configured to provide an optical function of not focusing an image on the retina of the eye of the wearer or an optical function of scattering incident light so as to suppress or slow down the progression of the abnormal refraction of the eye of the wearer; and/or
the functional area comprises an optical microstructure; and/or
the optical microstructure comprises a plurality of optical elements, such as for instance lenslets; and/or
the optical microstructure is disposed on an object-side surface and/or an eye-side surface and/or between the object-side surface and the eye-side surface of the lens; and/or
the plurality of optical elements is positioned randomly or in a network, preferably a regular network such as for instance a grid, a honeycomb, or concentric rings; and/or
the smallest distance between two adjacent and non-contiguous optical elements is ranging from 0.2 to 2 mm, preferably from 0.3 to 1.5 mm; and.
the smallest distance between two adjacent and non-contiguous optical elements is greater than or equal to 0.2 mm, preferably greater than or equal to 0.3 mm, and smaller than or equal to 2 mm, preferably smaller than or equal to 1.5 mm; and/or
the lens comprises a coating on an object-side surface and/or an eye-side surface; and/or
the lens comprises a coating on a surface comprising the functional area, for example on an object-side surface and/or an eye-side surface; and/or
the refractive area and/or the functional area is a connected area or a non-connected area; and/or
the optical function is different from the first refractive power (Rx); and/or
the functional area is not configured to provide the first refractive power (Rx).

According to a second aspect of the present invention, there also is provided a computer-implemented method for determining a lens intended to be worn in front of or on an eye of a wearer, to provide a first refractive power (Rx) based on a prescription of the wearer for correcting an abnormal refraction of the eye of the wearer, wherein the lens comprises a refractive area configured to provide the first refractive power (Rx) and a functional area configured to provide an optical function; wherein the method comprises the following steps:
providing a prescription of the wearer;
determining the refractive area so that on a first zone defined on the lens, more than 5%, for example more than 7%, of the surface of said first zone has a local refractive power ranging from the first refractive power (Rx) minus 0.25 diopter and the first refractive power (Rx) plus 0.25 diopter, and more than 25%, for example more than 30%, of the surface of said first zone covers the functional area.

According to a second aspect of the present invention, there also is provided a computer-implemented method for determining a spectacle lens intended to be worn in front of or on an eye of a wearer, to provide a first refractive power (Rx) based on a prescription of the wearer for correcting an abnormal refraction of the eye of the wearer, wherein the lens comprises a refractive area configured to provide the first refractive power (Rx) and on at least one surface a functional area configured to provide an optical function, and a coating covering the at least one surface having the optical functional area; wherein the method comprises the following steps:
providing a prescription of the wearer;
determining the refractive area so that on a first zone having a circular shape defined on the lens, more than 5%, for example more than 7% and less than 39% of the surface of said first zone has a local refractive power ranging from the first refractive power (Rx) minus 0.25 diopter and the first refractive power (Rx) plus 0.25 diopter, and more than 25%, for example more than 30%, and less than 60% of the surface of said first zone covers the functional area.

According to other embodiments of the second aspect, the proposed computer-implemented method may also comprise at least one of the following additional features:
more than 10% of the surface of said first zone has a local refractive power ranging from the first refractive power (Rx) minus 0.25 diopter and the first refractive power (Rx) plus 0.25 diopter; or more than 15% of the surface of said first zone has a local refractive power ranging from the first refractive power (Rx) minus 0.25 diopter and the first refractive power (Rx) plus 0.25 diopter; or more than 20% of the surface of said first zone has a local refractive power ranging from the first refractive power (Rx) minus 0.25 diopter and the first refractive power (Rx) plus 0.25 diopter; or more than 25% of the surface of said first zone has a local refractive power ranging from the first refractive power (Rx) minus 0.25 diopter and the first refractive power (Rx) plus 0.25 diopter.
more than 15% of the surface of said first zone has a local refractive power ranging from the first refractive power (Rx) minus 0.5 diopter and the first refractive power (Rx) plus 0.5 diopter; or more than 20% of the surface of said first zone has a local refractive power ranging from the first refractive power (Rx) minus 0.5 diopter and the first refractive power (Rx) plus 0.5 diopter; or more than 25% of the surface of said first zone has a local refractive power ranging from the first refractive power (Rx) minus 0.5 diopter and the first refractive power (Rx) plus 0.5 diopter; or more than 30% of the surface of said first zone has a local refractive power ranging from the first refractive power (Rx) minus 0.5 diopter and the first refractive power (Rx) plus 0.5 diopter; or more than 35% of the surface of said first zone has a local refractive power ranging from the first refractive power (Rx) minus 0.5 diopter and the first refractive power (Rx) plus 0.5 diopter.
less than 39% of the surface of said first zone has a local refractive power superior or equal to the first refractive power (Rx) plus 0.12 diopter; or less than 37% of the surface of said first zone has a local refractive power superior or equal to the first refractive power (Rx) plus 0.12 diopter; or less than 30% of the surface of said first zone has a local refractive power superior or equal to the first refractive power (Rx) plus 0.12 diopter.
the first zone is a circular zone having a diameter ranging from 6 mm to 10 mm.
the functional area has an optical function of not focusing an image on the retina of the eye of the wearer or an optical function of scattering incident light.
the functional area is configured to provide an optical function of not focusing an image on the retina of the eye of the wearer or an optical function of scattering incident light.
the functional area has an optical function of not focusing an image on the retina of the eye of the wearer or an optical function of scattering incident light so as to suppress or slow down the progression of the abnormal refraction of the eye of the wearer.
the functional area is configured to provide an optical function of not focusing an image on the retina of the eye of the wearer or an optical function of scattering incident light so as to suppress or slow down the progression of the abnormal refraction of the eye of the wearer.
the functional area comprises an optical microstructure.
the optical microstructure comprises a plurality of optical elements, such as for instance lenslets.
the optical microstructure is disposed on an object-side surface and/or an eye-side surface and/or between the object-side surface and the eye-side surface of the lens.
the plurality of optical elements is positioned randomly or in a network, preferably a regular network such as for instance a grid, a honeycomb, or concentric rings.
the smallest distance between two adjacent and non-contiguous optical elements is ranging from 0.2 to 2 mm, preferably from 0.3 to 1.5 mm.
the lens comprises a coating on an object-side surface and/or an eye-side surface.
the refractive area and/or the functional area is a connected area or a non-connected area.
the optical function is different from the first refractive power (Rx).
the functional area is not configured to provide the first refractive power (Rx).

According to a third aspect of the invention there is provided a computer program product comprising instructions for implementing a method according to the second aspect of the invention when the program is executed by a computer.

The is also provided a non-transitory storage medium readable by a computer storing instructions for implementing a method according to the second aspect of the invention, when executed by the computer.

### BRIEF DESCRIPTION OF THE DRAWINGS

The invention will be more clearly understood from the following description, given by way of example only, with reference to the accompanying drawings, in which:

FIG. 1 illustrates a plan view of a lens L1.

FIG. 2 illustrate a partial detailed view of a lens L1.

FIG. 3 illustrates a plan view of a lens L2.

FIG. 4 illustrate a partial detailed view of a lens L2.

FIG. 5 illustrates the percentage of surface having a local refractive power ranging within two ranges ([−0.25; 0.25] or [−0.50; 0.50]) centered on the first refractive power Rx for different lenses (lenses A to E).

FIG. 6 illustrates the visual acuity for the lenses A and B.

FIG. 7 illustrates the percentage of surface having a local refractive power (i) below the first refractive power Rx minus 0.12 diopter, or (ii) between the first refractive power Rx minus 0.12 diopter and the first refractive power Rx plus 0.12 diopter, or (iii) above the first refractive power Rx plus 0.12 diopter; for different lenses (lenses A to E).

FIG. 8 illustrates a general cross-section view of a lens.

### DETAILED DESCRIPTION OF THE INVENTION

To quantify the refractive power error in the refractive area of a lens, the inventors measured the surface of the lens having a local refractive power within a predefined range centred on the prescribed refractive power (Rx) for five different lenses. Each of said lens comprise a refractive area and a functional area configured to suppress or slow down myopia progression.

Among the five lenses on which measurements were carried out, four (lenses A, B, D and E) have the structure depicted in FIG. 1 (referred to as L1). L1 comprises a functional area comprising an optical microstructure 1 having a concentric ring configuration with lenslets 1. The geometry of the lenslets 1 has been calculated so as to generate a volume of non-focus light in front of the retina at any eccentricity, serving as myopia control. L1 further comprises a refractive area 2 comprising a central area, a peripheral area, and the area between the concentric rings of lenslets

The differences between lenses A, B, D and E are the following.

Lens A comprises spherical lenslets while lenses B, D and E comprise aspherical lenslets. Lenses A and B comprise 15 concentric rings of lenslets while lenses D and E comprise 11 concentric rings of lenslets. The ratio between the sum of surfaces of the lenslets and the surface of lens is about 50% for lenses A and B and about 40% for lenses D and E. Lenses A, B and D comprise lenslets on the object-side surface while lens E comprises lenslet between the object-side surface and the eye-side surface.

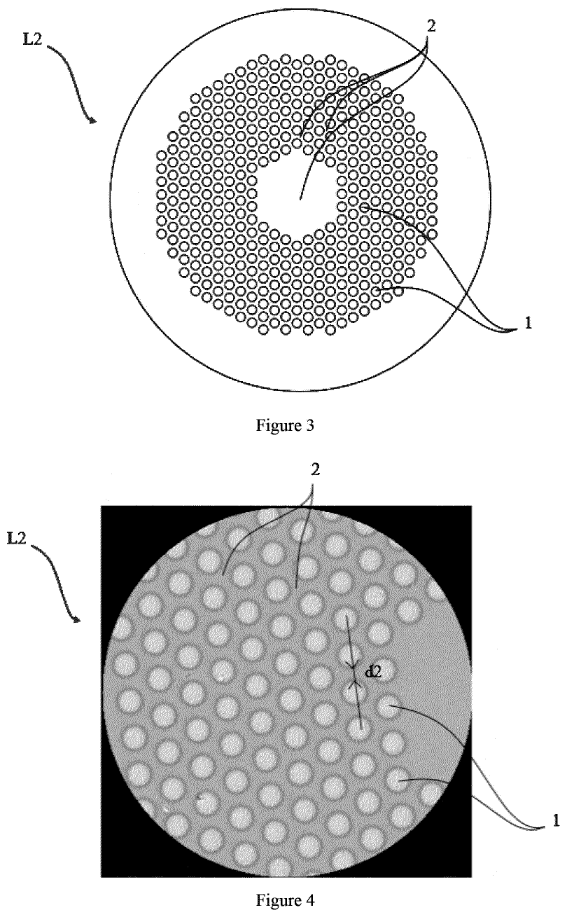

Among the five lenses on which measurements were carried out, one (lens C) has the structure depicted in FIG. 3 (referred to as L2). L2 comprises an optical microstructure 1 having an honeycomb configuration of spherical lenslets. The ratio between the sum of surfaces of the lenslets and the surface of lens is about 40%. The micro-lenses introduce myopic defocus at a plane in front of the retina by a relative positive power (+3.50 D). L2 comprises a connected refractive area while L1 comprises a non-connected refractive area.

The inventors carried out measurements of the local refractive power for lenses A, B, C, D and E. The local refractive power was measured using a NIMO TR1504 device. The measurements were carried out on coated lenses.

The NIMO TR1504 device is developed by Lambda-X and is available on the market.

Based on the measurements from the NIMO TR1504 device, the local refractive power is obtained as detailed below. The term local refractive power refers to the refractive power measured locally based on the numbers of pixels of a power map, especially a mean power map, derived from the measurements obtained with NIMO TR1504 device.

According to some embodiments, the local refractive power of a lens is obtained as follows:
a minimum power map and a cylinder map is obtained from the NIMO TR1504 device for the lens (saved in two separated “.csv” files).
the two files are computed using an image processing software, such as for instance ImageJ free software (https://imagej.nih.gov/ij/), to obtain a mean power map (Mean Power=Min Power+0.5*Cylinder).
The mean power map is filtered using the 3×3 pixel “smooth” command of the image processing software, here ImageJ. The inventors have verified than filtering limits the noise without changing the results.
The local refractive power corresponds to the value of the mean power of a pixel of the mean power map, preferably the filtered mean power map.

According to some embodiments, the first refractive power (Rx) is based on the measurements of the mean power map, preferably the filtered mean power map, at the center of the lens. The center of the lens may be the geometrical center of the lens or the optical center of the lens.

According to some embodiments, the first refractive power (Rx) is the mean value of the values of the mean power map, preferably the filtered mean power map, at the center of the lens inside a circular window of a few millimetres, for example having a diameter of a few millimetres, (for instance from 1 to 7 millimetres or from 1 to 5 millimetres or from 1 to 3 millimetres).

According to some embodiments, the percentage of surface having a local refractive power inside a predefined range is computed based on the number of pixels of the mean power map, preferably the filtered mean power map, of a measurement zone for which the value of the mean power is inside the predefined range compared to the total number of pixels inside the measurement zone.

The inventors also carried out measurement of the visual performance of the lenses. FIG. 6 shows the logMAR chart for the lenses A and B. The use of LogMAR (Logarithm of the Minimum Angle of Resolution) for measuring visual acuity is well known to the skilled artisan. The results depicted in FIG. 6 are based on subjective measurements from 6 participants in different test conditions. As it is apparent from the results, lens A exhibits the lowest visual performance. Therefore, the inventors considered that the values of lens A could be considered as a lower limit.

In view of the above, the inventors determined the following lower limits of values for the measurement zone:
more than 5%, for example more than 7%, of the surface has a local refractive power ranging from the refractive power (Rx) minus 0.25 diopter and the first refractive power (Rx) plus 0.25 diopter.
more than 15% of the surface has a local refractive power ranging from the first refractive power (Rx) minus 0.5 diopter and the first refractive power (Rx) plus 0.5 diopter.
   The measurement zone (also referred to as the first zone) is a zone defined on the lens so that at least 30% of the surface of said zone covers the functional area of the lens and less than 60% of the surface of said first zone covers the functional area. It means that the measures are carried out on a zone which extends over the lens so that at least 30% of the surface of the zone extends over the functional area of the lens and less than 60% of the surface of said first zone covers the functional area

The first zone may be a circular zone having a diameter greater than or equal to 6 mm and smaller than or equal to 10 mm.

For example, in the NIMO TR1504 device, the measurement zone is a circular zone having a diameter ranging from 6 mm to 10 mm.

According to a first aspect, the invention relates to a lens intended to be worn by a wearer and to provide a first refractive power (Rx) based on a prescription of the wearer for correcting an abnormal refraction of the eye of the wearer.

According to some embodiments, the lens is a spectacle lens intended to be worn in front of an eye of a wearer. According to some embodiments, the lens is a contact lens intended to be worn on an eye of a wearer.

The lens comprises a refractive area configured to provide the first refractive power (Rx) for correcting the abnormal refraction of the eye of the wearer and a functional area configured to provide an optical function. According to some embodiments, the functional area is not configured to provide the first refractive power (Rx). According to some embodiments, the functional area is not configured to provide only the first refractive power (Rx). It means that the functional area may be configured to provide the first refractive power (Rx), or a second refractive power different from the first refractive power, together with an additional optical function. According to some embodiments, the functional area is configured to provide an optical function different from the first refractive power (Rx). According to some embodiments, the lens consists of the refractive area and the functional area.

According to some embodiments, the functional area is defined has any area having a local refractive power above the first refractive power (Rx) plus 1 diopter or below the first refractive power (Rx) minus 1 diopter.

For example, the lens according to the invention is arranged for myopia control and the functional area is defined has any area having a local refractive power above the first refractive power (Rx) plus 0.75 diopter, for example plus 1 diopter for example plus 2 diopters.

For example, the lens according to the invention is arranged for hypermetropia control and the functional area is defined has any area having a local refractive power above the first refractive power (Rx) minus 0.75 diopter, for example minus 1 diopter for example minus 2 diopters.

According to some embodiments, the functional area is defined has any area wherein the difference between the local refractive power and the first refractive power (Rx) is above 1 diopter in absolute value, i.e. |Rx−LRP|>1D, with LRP the local refractive power and Rx the first refractive power.

According to some embodiments, the first refractive power (Rx) is based on a prescription of the wearer for correcting an abnormal refraction of the eye of the wearer in standard wearing conditions. According to some embodiments, the first refractive power (Rx) corresponds to the refractive power prescribed by the eye-care practitioner. According to some embodiments, the first refractive power (Rx) corresponds to the refractive power at the center of the lens.

According to some embodiments, the first refractive power (Rx) is based on the measurements of the mean power map, preferably the filtered mean power map, at the center of the lens. According to some embodiments, the first refractive power (Rx) is the mean value of the values of the mean power map, preferably the filtered mean power map, at the center of the lens inside a window of a few millimetres, for example a diameter of a few millimeters (for instance from 1 to 7 millimetres or from 1 to 5 millimetres or from 1 to 3 millimetres).

According to some embodiments, the functional area has an optical function of not focusing an image on the retina of the eye of the wearer, an optical function of scattering incident light or an optical function of focusing an image other than on the retina of the eye of the wearer, for example when considering an Atchison eye model.

According to some embodiments, the functional area comprises an optical microstructure having an optical function of not focusing an image on the retina of the eye of the wearer, for example when considering an Atchison eye model, so as to slow down progression of the abnormal refraction of the eye of the wearer. In the sense of the invention “focusing” is to be understood as producing a focusing spot with a circular section that can be reduced to a point in the focal plane. Advantageously, such optical function of the optical element reduces the deformation of the retina of the eye of the wearer in peripheral vision, allowing to slow down the progression of the abnormal refraction of the eye of the person wearing the lens. According to some embodiments, the lens comprises an optical microstructure having an optical function of focusing an image other than on the retina of the eye of the wearer so as to slow down progression of the abnormal refraction of the eye of the wearer.

According to some embodiments, the lens comprises an optical microstructure having an optical function of not focusing an image on the retina of the eye of the wearer, for example when considering an Atchison eye model, in standard wearing conditions so as to slow down progression of the abnormal refraction of the eye of the wearer. According to some embodiments, the lens comprises an optical microstructure having an optical function of focusing an image other than on the retina of the eye of the wearer in standard wearing conditions so as to slow down progression of the abnormal refraction of the eye of the wearer.

According to some embodiments, the functional area comprises an optical microstructure having an additional optical function of providing the first refractive power (Rx) based on the prescription of the wearer for correcting the abnormal refraction of the eye of the wearer in standard wearing conditions.

More than 5%, for example more than 7%, of the surface of the first zone has a local refractive power ranging from the first refractive power (Rx) minus 0.25 diopter and the first refractive power (Rx) plus 0.25 diopter. It means that the refractive power error is lower than 0.25 diopter for at least 5%, for example at least 7% of the surface of the first zone. The first zone is a measurement zone defined on the lens so that at least 30% of the surface of said zone covers the functional area of the lens. According to some embodiment, the first zone is defined on the lens so that at least 35% of the surface of said zone covers the functional area of the lens. According to some embodiment, the first zone is defined on the lens so that at least 40% of the surface of said zone covers the functional area of the lens. According to some embodiment, the first zone is any zone defined on the lens so that at least 30%, at least 35% or at least 40% of the surface of said zone covers the functional area of the lens.

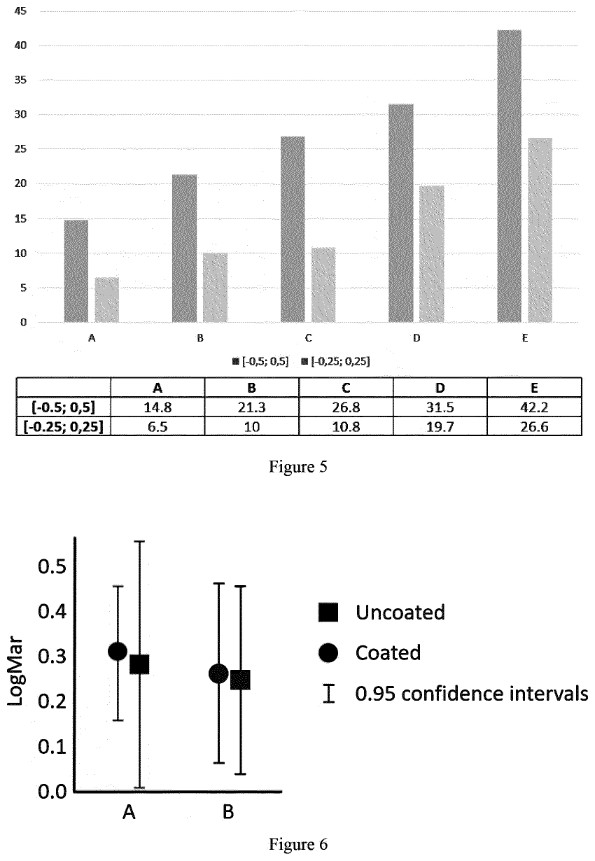

As illustrated in FIG. 5, the inventors have measured that the value of percentage of surface of the first zone having a local refractive power ranging from the first refractive power (Rx) minus 0.25 diopter and the first refractive power (Rx) plus 0.25 diopter is equal to 6.5 for lens A.

According to some embodiments, more than 10% of surface of the first zone has a local refractive power ranging from the first refractive power (Rx) minus 0.25 diopter and the first refractive power (Rx) plus 0.25 diopter. As illustrated in FIG. 5, the percentage of surface of the first zone having a local refractive power ranging from the first refractive power (Rx) minus 0.25 diopter and the first refractive power (Rx) plus 0.25 diopter is equal to 10 for lens B.

According to some embodiments, more than 15% of surface of the first zone has a local refractive power ranging from the first refractive power (Rx) minus 0.25 diopter and the first refractive power (Rx) plus 0.25 diopter. As illustrated in FIG. 5, the percentage of surface of the first zone having a local refractive power ranging from the first refractive power (Rx) minus 0.25 diopter and the first refractive power (Rx) plus 0.25 diopter is equal to 10.8 for lens C.

According to some embodiments, more than 20% of surface of the first zone has a local refractive power ranging from the first refractive power (Rx) minus 0.25 diopter and the first refractive power (Rx) plus 0.25 diopter. As illustrated in FIG. 5, the percentage of surface of the first zone having a local refractive power ranging from the first refractive power (Rx) minus 0.25 diopter and the first refractive power (Rx) plus 0.25 diopter is equal to 19.7 for lens D.

According to some embodiments, more than 25% of surface of the first zone has a local refractive power ranging from the first refractive power (Rx) minus 0.25 diopter and the first refractive power (Rx) plus 0.25 diopter. As illustrated in FIG. 5, the percentage of surface of the first zone having a local refractive power ranging from the first refractive power (Rx) minus 0.25 diopter and the first refractive power (Rx) plus 0.25 diopter is equal to 26.6 for lens E. According to some embodiments, more than 30% of surface of the first zone has a local refractive power ranging from the first refractive power (Rx) minus 0.25 diopter and the first refractive power (Rx) plus 0.25 diopter.

According to some embodiments, more than 15% of the surface of the first zone has a local refractive power ranging from a first refractive power (Rx) minus 0.5 diopter and the first refractive power (Rx) plus 0.5 diopter. It means that the refractive power error is lower than 0.5 diopter for at least 15% of the surface of the first zone.

As illustrated in FIG. 5, the inventors have measured that the value of percentage of surface of the first zone having a local refractive power ranging from the first refractive power (Rx) minus 0.5 diopter and the first refractive power (Rx) plus 0.5 diopter is equal to 14.8 for lens A.

According to some embodiments, more than 20% of surface of the first zone has a local refractive power ranging from the first refractive power (Rx) minus 0.5 diopter and the first refractive power (Rx) plus 0.5 diopter. As illustrated in FIG. 5, the percentage of surface of the first zone having a local refractive power ranging from the first refractive power (Rx) minus 0.5 diopter and the first refractive power (Rx) plus 0.5 diopter is equal to 21.3 for lens B.

According to some embodiments, more than 25% of surface of the first zone has a local refractive power ranging from the first refractive power (Rx) minus 0.5 diopter and the first refractive power (Rx) plus 0.5 diopter. As illustrated in FIG. 5, the percentage of surface of the first zone having a local refractive power ranging from the first refractive power (Rx) minus 0.5 diopter and the first refractive power (Rx) plus 0.5 diopter is equal to 26.8 for lens C.

According to some embodiments, more than 30% of surface of the first zone has a local refractive power ranging from the first refractive power (Rx) minus 0.5 diopter and the first refractive power (Rx) plus 0.5 diopter. As illustrated in FIG. 5, the percentage of surface of the first zone having a local refractive power ranging from the first refractive power (Rx) minus 0.5 diopter and the first refractive power (Rx) plus 0.5 diopter is equal to 31.5 for lens D.

According to some embodiments, more than 35% of surface of the first zone has a local refractive power ranging from the first refractive power (Rx) minus 0.5 diopter and the first refractive power (Rx) plus 0.5 diopter. According to some embodiments, more than 40% of surface of the first zone has a local refractive power ranging from the first refractive power (Rx) minus 0.5 diopter and the first refractive power (Rx) plus 0.5 diopter. As illustrated in FIG. 5, the percentage of surface of the first zone having a local refractive power ranging from the first refractive power (Rx) minus 0.5 diopter and the first refractive power (Rx) plus 0.5 diopter is equal to 42.2 for lens E.

According to some embodiments, more than 45% of surface of the first zone has a local refractive power ranging from the first refractive power (Rx) minus 0.5 diopter and the first refractive power (Rx) plus 0.5 diopter.

According to some embodiments, less than 80% of surface of the first zone has a local refractive power ranging from the first refractive power (Rx) minus 0.25 diopter and the first refractive power (Rx) plus 0.25 diopter. According to some embodiments, less than 90% of surface of the first zone has a local refractive power ranging from the first refractive power (Rx) minus 0.5 diopter and the first refractive power (Rx) plus 0.5 diopter.

The inventors also measured the surface of the lens having a local refractive power superior or equal to the first refractive power (Rx) plus 0.12 diopter. For said measurement, the local refractive power is lower than the first refractive power (Rx) plus 40 diopters.

According to some embodiments, less than 39% of the surface of the first zone has a local refractive power superior or equal to the first refractive power (Rx) plus 0.12 diopter.

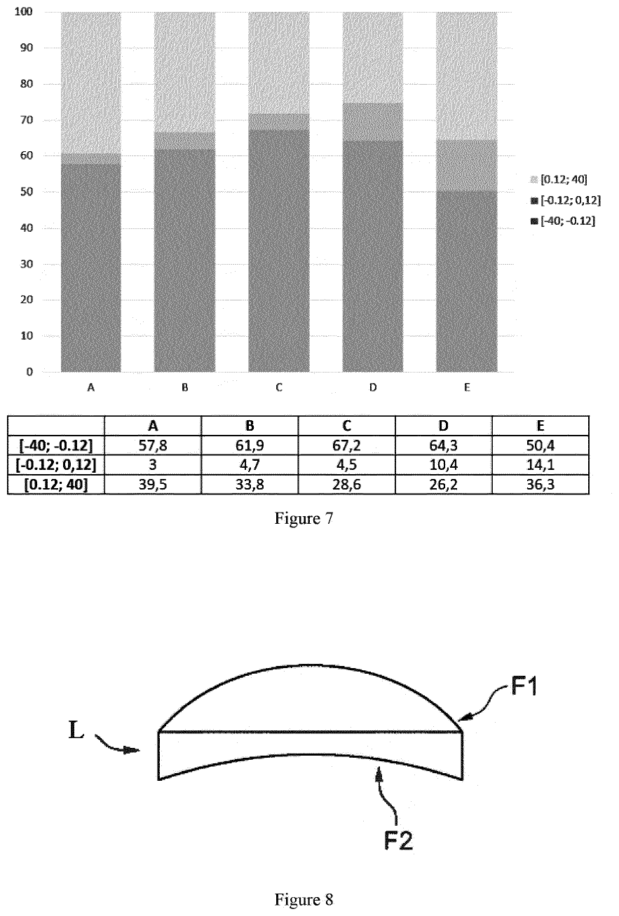

As illustrated in FIG. 7, the inventors have measured that the value of percentage of surface of the first zone having a local refractive power superior or equal to the first refractive power (Rx) plus 0.12 diopter is equal to 39.5 for lens A, 36.3 for lens E, 33.8 for lens B, 28.6 for lens C and 26.2 for lens D.

According to some embodiments, less than 37% of the surface of the first zone has a local refractive power superior or equal to the first refractive power (Rx) plus 0.12 diopter. According to some embodiments, less than 30% of the surface of the first zone has a local refractive power superior or equal to the first refractive power (Rx) plus 0.12 diopter. According to some embodiments, less than 29% of the surface of the first zone has a local refractive power superior or equal to the first refractive power (Rx) plus 0.12 diopter. According to some embodiments, less than 28% of the surface of the first zone has a local refractive power superior or equal to the first refractive power (Rx) plus 0.12 diopter. According to some embodiments, less than 26% of the surface of the first zone has a local refractive power superior or equal to the first refractive power (Rx) plus 0.12 diopter.

The inventors also measured the surface of the lens having a local refractive power inferior or equal to the first refractive power (Rx) minus 0.12 diopter. For said measurement, the local refractive power is higher than the first refractive power (Rx) minus 40 diopters.

According to some embodiments, less than 70% of the surface of the first zone has a local refractive power inferior or equal to the first refractive power (Rx) minus 0.12 diopter.

As illustrated in FIG. 7, the inventors have measured that the value of percentage of surface of the first zone having a local refractive power inferior or equal to the first refractive power (Rx) minus 0.12 diopter is equal to 67.2 for lens C, 64.3 for lens D, 61.9 for lens B, 57.8 for lens A and 50.4 for lens E.

According to some embodiments, less than 68% of the surface of the first zone has a local refractive power inferior or equal to the first refractive power (Rx) minus 0.12 diopter. According to some embodiments, less than 65% of the surface of the first zone has a local refractive power inferior or equal to the first refractive power (Rx) minus 0.12 diopter. According to some embodiments, less than 62% of the surface of the first zone has a local refractive power inferior or equal to the first refractive power (Rx) minus 0.12 diopter. According to some embodiments, less than 60% of the surface of the first zone has a local refractive power inferior or equal to the first refractive power (Rx) minus 0.12 diopter. According to some embodiments, less than 55% of the surface of the first zone has a local refractive power inferior or equal to the first refractive power (Rx) minus 0.12 diopter. According to some embodiments, less than 50% of the surface of the first zone has a local refractive power inferior or equal to the first refractive power (Rx) minus 0.12 diopter.

The inventors also measured the surface of the lens having a local refractive power ranging from the first refractive power (Rx) minus 0.12 diopter and the first refractive power (Rx) plus 0.12 diopter.

According to some embodiments, more than 3% of the surface of the first zone has a local refractive power ranging from the first refractive power (Rx) plus 0.12 diopter and the first refractive power (Rx) minus 0.12 diopter.

As illustrated in FIG. 7, the inventors have measured that the value of percentage of surface of the first zone having a local refractive power ranging from the first refractive power (Rx) plus 0.12 diopter and the first refractive power (Rx) minus 0.12 diopter is equal to 3 for lens A, 4.7 for lens B, 4.5 for lens C, 10.4 for lens D and 14.1 for lens E.

According to some embodiments, more than 3% of the surface of the first zone has a local refractive power ranging from the first refractive power (Rx) plus 0.12 diopter and the first refractive power (Rx) minus 0.12 diopter. According to some embodiments, more than 4% of the surface of the first zone has a local refractive power ranging from the first refractive power (Rx) plus 0.12 diopter and the first refractive power (Rx) minus 0.12 diopter. According to some embodiments, more than 4.5% of the surface of the first zone has a local refractive power ranging from the first refractive power (Rx) plus 0.12 diopter and the first refractive power (Rx) minus 0.12 diopter. According to some embodiments, more than 5% of the surface of the first zone has a local refractive power ranging from the first refractive power (Rx) plus 0.12 diopter and the first refractive power (Rx) minus 0.12 diopter. According to some embodiments, more than 10% of the surface of the first zone has a local refractive power ranging from the first refractive power (Rx) plus 0.12 diopter and the first refractive power (Rx) minus 0.12 diopter. According to some embodiments, more than 13% of the surface of the first zone has a local refractive power ranging from the first refractive power (Rx) plus 0.12 diopter and the first refractive power (Rx) minus 0.12 diopter. According to some embodiments, more than 14% of the surface of the first zone has a local refractive power ranging from the first refractive power (Rx) plus 0.12 diopter and the first refractive power (Rx) minus 0.12 diopter. According to some embodiments, more than 15% of the surface of the first zone has a local refractive power ranging from the first refractive power (Rx) plus 0.12 diopter and the first refractive power (Rx) minus 0.12 diopter.

According to some embodiments, more than 10% of surface of the first zone has a local refractive power ranging from the first refractive power (Rx) minus 0.25 diopter and the first refractive power (Rx) plus 0.25 diopter. According to some embodiments, more than 25% of surface of the first zone has a local refractive power ranging from the first refractive power (Rx) minus 0.5 diopter and the first refractive power (Rx) plus 0.5 diopter. According to some embodiments, less than 30% of the surface of the first zone has a local refractive power superior or equal to the first refractive power (Rx) plus 0.12. According to some embodiments, less than 70% of the surface of the first zone has a local refractive power inferior or equal to the first refractive power (Rx) minus 0.12 diopter. According to some embodiments, more than 4% of the surface of the first zone has a local refractive power ranging from the first refractive power (Rx) plus 0.12 diopter and the first refractive power (Rx) minus 0.12 diopter.

According to some embodiments represented for instance on FIG. 1 or 3, a lens comprises an optical microstructure 1 having an optical function. According to some embodiments, the optical microstructure has the optical function of not focusing an image on the retina of the eye of the wearer in standard wearing conditions so as to slow down progression of the abnormal refraction of the eye of the wearer.

According to some embodiments, the optical microstructure 1 has an additional optical function of providing the refractive power based on the prescription of the wearer for correcting the abnormal refraction of the eye of the wearer.

The lens further comprises a refractive area 2 configured to provide the refractive power based on the prescription of the wearer for correcting the abnormal refraction of the eye of the wearer. The refractive area is preferably formed as the area other than optical microstructure. In other words, the refractive area is the complementary area to the area formed by the optical microstructure. According to some embodiments, the central area of the lens does not comprise any optical element. For example, the lens may comprise an empty zone centred on the optical center of said lens and having a diameter equal to 9 mm which does not comprise any optical element. The optical center of the lens may correspond to the fitting point of the lens. Alternatively, the optical elements may be disposed on the entire surface of the lens. According to some embodiments, the refractive area is further configured to provide to the wearer, in particular for foveal vision, a second refractive power different from the first refractive power based on the prescription of the wearer. In the sense of the invention, the two refractive powers are considered different when the difference between the two refractive powers is greater than or equal to 0.5 diopter.

The term “prescription” is to be understood to mean a set of optical characteristics of refractive power, of astigmatism, of prismatic deviation, determined by an eye care practitioner such as an ophthalmologist or optometrist in order to correct the vision defects of the eye, for example by means of a spectacle lens positioned in front of the eye or by means of a contact lens positioned on the eye. For example, the prescription for a myopic eye comprises the values of refractive power and of astigmatism with an axis for the distance vision.

As illustrated on FIG. 8, a lens L according to the invention comprises an object-side surface F1 formed as a convex curved surface toward an eye-side surface F2 formed as a concave surface having a different curvature than the curvature of the object side surface F1.

According to some embodiments, the lens comprises a coating on the object-side surface and/or the eye-side surface. According to said embodiments, the local refractive power is measured with or without the coating.

According to an embodiment, the lens may comprise at least one layer of at least one coating element. The at least one layer of at least one coating element covers at least part of the surface of the lens having the functional area.

The at least one layer of at least one coating element may be characterized by different parameters such as an index of refraction and a thickness. The coating layer is also defined by a coating process characterized by different parameters such as for example the curing time or the temperature and/or viscosity of the coating element during the coating operations.

The at least one layer of at least one coating element is characterized by a refractive index and a local thickness, and thus participates to the optical function of the functional area.

Moreover, when the at least one layer of at least one coating element is applied on the lens, the viscosity of the at least one coating element combined with the complex shape of the surface of the lens comprising functional area may result in a non-homogenous repartition of said at least one coating element over the surface of the lens.

According to some embodiments, the optical microstructure is disposed on the object-side surface and/or the eye-side surface and/or between the object-side surface and the eye-side surface of the lens.

According to some embodiments, the optical microstructure modifies the intensity and/or curvature of the wavefront and/or deviates light.

According to some embodiments, the optical microstructure modifies the wavefront curvature in a range from −20 diopters to +20 diopters, preferably in a range from −10 diopters to +10 diopters, more preferably in a range from −5.5 diopters to 5.5 diopters. According to some embodiments, the optical microstructure modifies the wavefront curvature locally, i.e., at the intersection between the optical microstructure and the wavefront.

According to an embodiment, the lens according to the invention is arranged for myopia control and the optical microstructure modifies the wavefront curvature of at least +0.75 diopter, for example of at least +1 diopter, for example of at least +2 diopters, for example of at least +5.5 diopters, for example of at least +10 diopters, for example of at least +20 diopters.

According to an embodiment, the lens according to the invention is arranged for hypermetropia control and the optical microstructure modifies the wavefront curvature of at least −0.75 diopter, for example of at least −1 diopter, for example of at least −2 diopters, for example of at least −5.5 diopters, for example of at least −10 diopters, for example of at least −20 diopters.

According to some embodiments, the optical microstructure scatters light. The light may be scattered with an angle ranging from +/−1° to +/−30°. According to some embodiments, the optical microstructure scatters light locally, i.e., at the intersection between the optical microstructure and the wavefront.

According to some embodiments, the ratio between the surface of the optical microstructure and the surface of said lens is comprised between 20% and 70%, preferably between 30% and 60%, and more preferably between 40% and 50%.

According to some embodiments, the optical microstructure comprises a plurality of optical elements, such as for instance lenslets.

According to some embodiments, at least one, at least 50%, at least 80% and preferably all the optical elements has an optical function of not focusing an image on the retina of the eye of the wearer. According to some embodiments, at least one, at least 50%, at least 80% and preferably all the optical elements has an optical function of not focusing an image on the retina of the eye of the wearer in standard wearing conditions. According to some embodiments, at least one, at least 50%, at least 80%, more preferably all, of the optical elements have an optical function of focusing an image for peripheral vision on a position other than the retina. According to some embodiments, at least one, at least 50%, at least 80%, more preferably all, of the optical elements have an optical function of focusing an image for peripheral vision on a position other than the retina in standard wearing conditions.

According to a preferred embodiment of the invention, all of the optical elements are configured so that the mean focus of the light rays passing through each optical element is at a same distance to the retina of the wearer, at least for peripheral vision. The optical function, in particular the dioptric function, of each optical element may be optimized so as to provide a focus image, in particular in peripheral vision, at a constant distance of the retina of the eye of the wearer. Such optimization requires adapting the dioptric function of each of the optical element depending on their position on the lens. The optical element density or the quantity of power may be adjusted depending on zones of the lens. Typically, the optical element may be positioned in the periphery of the lens, in order to increase the effect of the optical element on myopia control, so as to compensate peripheral defocus due to the peripheral shape of the retina.

According to a preferred embodiment of the invention, the optical elements are independent. In the sense of the invention, two optical elements are considered as independent if producing independent images. In particular, when illuminated by a parallel beam “in central vision”, each “independent contiguous optical element” forms on a plane in the image space a spot associated with it.

According to some embodiments, the optical elements have a height ranging from 0.1 μm to 50 μm. According to some embodiments, the optical elements have a length ranging from 0.5 μm to 1.5 mm. According to some embodiments, the optical elements have width ranging from 0.5 μm to 1.5 mm. According to some embodiments, the optical elements have a diameter ranging from 0.5 mm to 3.0 mm, preferably from 0.8 mm to 2.0 mm, more preferably from 1.0 mm to 1.2 mm. According to some embodiments, the optical elements have a contour shape being inscribable in a circle having a diameter greater than or equal to 0.5 mm and smaller than or equal to 3.0 mm, preferably greater than or equal to 1.0 mm and smaller than 2.0 mm. According to some embodiments, the optical elements introduce myopic defocus at a plane in front of the retina by a relative positive power ranging from 2 to 7 diopters, preferably ranging from 3.5 to 5.5 diopters.

According to some embodiments, the ratio between the sum of areas of the optical elements and the area of lens is greater or equal to 20%, for example greater than or equal to 30%, for example greater than or equal to 40%, and smaller than or equal to 70%, for example smaller than or equal to 60%, for example smaller than or equal to 50%.

According to some embodiments, the plurality of optical elements is positioned in a network. The network may be a random network or a structured network, such as for instance a grid, a honeycomb, or concentric rings. According to some embodiments, the structured network is a squared network, a hexagonal network, a triangle network, or an octagonal network.

According to some embodiments, the plurality of optical elements is positioned in a grid with a constant grid step.

According to some embodiments, the smallest distance between two adjacent and non-contiguous optical elements is ranging from 0.2 to 2 mm or from 0.3 to 1.5 mm.

According to some embodiments, the smallest distance between two adjacent and non-contiguous optical elements is ranging from 1 to 1.5 mm. The term smallest distance is to be understood as the smallest distance between the periphery of two adjacent and non-contiguous optical elements.

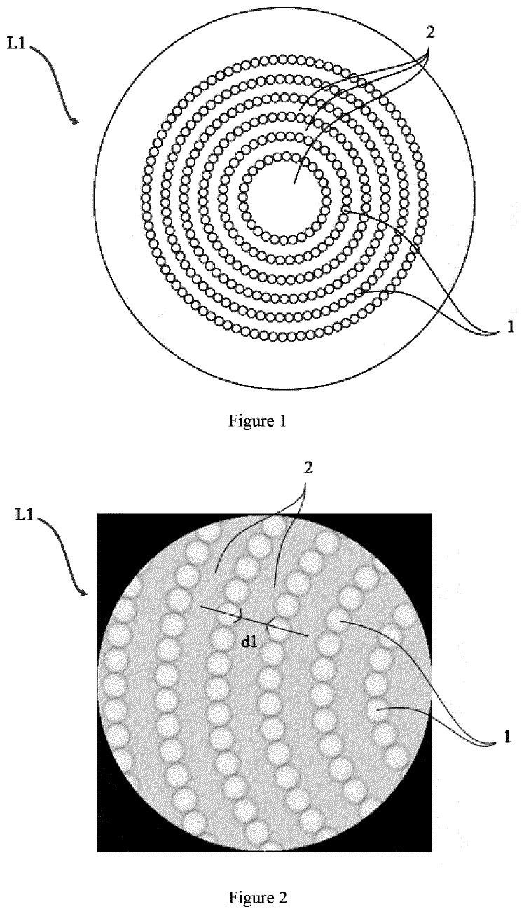

The smallest distance between two adjacent and non-contiguous optical elements d1 is depicted in FIG. 2 for a lens having concentric rings lenslets. The smallest distance between two adjacent and non-contiguous optical elements d2 is depicted in FIG. 4 for a lens having an honeycomb configuration of lenslets. According to some embodiments, d1 is greater than or equal to 0.2 mm, for example greater than or equal to 0.5 mm, for example greater than or equal to 0.6 mm and smaller than or equal to 2 mm, for example smaller than or equal to 1.5 mm, for example smaller than or equal to 1.4 mm.

According to some embodiments wherein the number of concentric rings is above 13, d1 is greater than or equal to 0.2 mm, for example greater than or equal to 0.4 mm, for example greater than or equal to 0.5 mm, for example greater than or equal to 0.6 mm and smaller than or equal to 2 mm, for example smaller than or equal to 1 mm, for example smaller than or equal to 0.9 mm, for example smaller than or equal to 0.8 mm. According to some embodiments wherein the number of concentric rings is below 13, d1 is greater than or equal to 0.2 mm, for example greater than or equal to 0.8 mm, for example greater than or equal to 1 mm and smaller than or equal to 2 mm, for example smaller than or equal to 1.6 mm, for example smaller than or equal to 1.5 mm. According to some embodiments, d2 is greater than or equal to 0.2 mm, for example greater than or equal to 0.3 mm, for example greater than or equal to 0.4 mm and smaller than or equal to 2 mm, for example smaller than or equal to 1 mm, for example smaller than or equal to 0.8 mm, for example smaller than or equal to 0.6 mm.

According to some embodiments, the smallest distance between a pair of adjacent and non-contiguous optical elements is constant for each pair of adjacent and non-contiguous optical elements.

According to some embodiments, at least part of the optical elements is contiguous. In the sense of the present invention, two optical elements located on a surface of the lens are contiguous if there is a path supported by said surface that links the two optical elements and if along said path one does not reach the basis surface on which the optical elements are located. When the surface on which the at least two optical elements are located is spherical, the basis surface corresponds to said spherical surface. In other words, two optical elements located on a spherical surface are contiguous if there is a path supported by said spherical surface and linking them and if along said path one may not reach the spherical surface.

When the surface on which the at least two optical elements are located is non-spherical, the basis surface corresponds to the local spherical surface that best fit said non-spherical surface. In other words, two optical elements located on a non-spherical surface are contiguous if there is a path supported by said non-spherical surface and linking them and if along said path one may not reach the spherical surface that best fit the non-spherical surface.

According to some embodiments, the contiguous optical elements are independent.

According to some embodiment, the distance between optical elements ranges from 0 (contiguous optical elements) and 3 times the length and/or width of the optical elements.

According to some embodiments, at least one, for example all, of the optical elements has a non-spherical optical function. According to some embodiments, at least one, for example all, of the optical elements has an aspherical optical function.

According to some embodiments, at least one, for example all, of the optical elements has a spherical optical function.

According to some embodiments, at least one of the optical elements, preferably more than 50%, more preferably more than 80% of the optical elements are aspherical lenslets. In the sense of the invention, aspherical lenslets have a continuous power evolution over their surface. An aspherical lenslet may have an asphericity comprised between 0.1 diopter and 3 diopters. The asphericity of an aspherical lenslet corresponds to the difference of refractive power measured in the center of the lenslet and the refractive power measured in the periphery of the lenslet.

The center of the lenslet may be defined by a spherical area centered on the geometrical center of the lenslet and having a diameter comprised between 0.1 mm and 0.5 mm, preferably equal to 2.0 mm. The periphery of the lenslets may be defined by an annular zone centered on the geometrical center of the lenslets and having an inner diameter comprised between 0.5 mm and 0.7 mm and an outer diameter comprised between 0.70 mm and 0.80 mm.

According to some embodiments, the aspherical lenslets have a refractive power in their geometrical center comprised between 2.0 diopters and 7 diopters in absolute value, and a refractive power in their periphery comprised between 1.5 diopters and 6.0 diopters in absolute value.

The asphericity of the different aspherical lenslets before the coating of the surface of the lens on which the optical elements are disposed may vary from on lenslet to the other according to the radial distance from the optical center of said lens.

Additionally, the asphericity of the different aspherical lenslets after the coating of the surface of the lens on which the optical elements are disposed may further vary from on lenslet to the other according to the radial distance from the optical center of said lens

According to some embodiments, at least part, for example all, of the optical elements have a varying refractive power and a continuous first derivative between two contiguous optical elements. According to some embodiments, at least part, for example all, of the optical elements have a constant refractive power and a discontinuous first derivative between two contiguous optical elements.

The optical elements can be made using different technologies such as non limitatively direct surfacing, molding, casting, injection, embossing, filming, or photolithography.

According to some embodiments, the plurality of optical elements is positioned as detailed in WO2019/166659. According to some embodiments, the plurality of optical elements is positioned along a plurality of concentric rings.

According to some embodiments, the lens comprises at least four optical elements organized in at least two groups of contiguous optical elements.

According to some embodiments, each group of contiguous optical element is organized in at least two concentric rings having the same center, the concentric ring of each group of contiguous optical element being defined by an inner diameter corresponding to the smallest circle that is tangent to at least one optical element of said group and an outer diameter corresponding to the largest circle that is tangent to at least one optical element of said group.

According to some embodiments, at least part of, for example all the concentric rings of optical elements are centered on the optical center of the surface of the lens on which said optical elements are disposed.

According to some embodiments, the concentric rings of optical elements have a diameter comprised between 9.0 mm and 60 mm. According to some embodiments, considering an annular zone of the lens having an inner diameter greater than 9 mm and an outer diameter smaller than 60 mm, having a geometrical center located at a distance of the optical center of the lens smaller than 1 mm, the ratio between the sum of areas of the parts of optical elements located inside said circular zone and the area of said circular zone is comprised between 20% and 70%, preferably between 30% and 60%, and more preferably between 40% and 50%.

According to some embodiments, the distance between two successive concentric rings of optical elements is greater than or equal to 2.0 mm, 3.0 mm or 5.0 mm, the distance between two successive concentric rings being defined by the difference between the outer diameter of a first concentric ring and the inner diameter of a second concentric ring, the second concentric ring being closer to the periphery of the lens. Advantageously, having the distance between two successive concentric rings of optical elements greater than 2.0 mm allows managing a larger refractive area between these rings of optical elements and thus provides better visual acuity.

According to some embodiments, the lens comprises optical elements disposed in at least 2 concentric rings, preferably more than 5, more preferably more than 10 concentric rings. For example, the optical elements may be disposed in 11 or 15 concentric rings centered on the optical center of the lens. The refractive power and/or cylinder of the lenslets may be different depending on their position along the concentric rings.

According to some embodiments, the optical elements are configured so that along at least one section of the lens the mean sphere of optical elements increases from a point of said section towards the peripheral part of said section.

According to some embodiments, the optical elements are configured so that along at least one section of the lens the cylinder of optical elements increases from a point of said section towards the peripheral part of said section.

According to some embodiments, the optical elements are configured so that along the at least one section of the lens the mean sphere and/or the cylinder of optical elements increases from the center of said section towards the peripheral part of said section.

According to some embodiments, the refractive area comprises an optical center and the optical elements are configured so that along any section passing through the optical center of the lens the mean sphere and/or the cylinder of the optical elements increases from the optical center towards the peripheral part of the lens.

According to some embodiments, the refractive area comprises a far vision reference point, a near vision reference point, and a meridian joining the far and near vision reference points, the optical elements are configured so that in standard wearing conditions along any horizontal section of the lens the mean sphere and/or the cylinder of the optical elements increases from the intersection of said horizontal section with the meridian towards the peripheral part of the lens.

According to some embodiments, the mean sphere and/or the cylinder increase function along the sections are different depending on the position of said section along the meridian.

According to some embodiments, the mean sphere and/or the cylinder increase function along the sections are unsymmetrical.

According to some embodiments, the optical elements are configured so that in standard wearing conditions the at least one section is a horizontal section.

According to some embodiments, the mean sphere and/or the cylinder of optical elements increases from a first point of said section towards the peripheral part of said section and decreases from a second point of said section towards the peripheral part of said section, the second point being closer to the peripheral part of said section than the first point.

According to some embodiments, the mean sphere and/or the cylinder increase function along the at least one section is a Gaussian function.

According to some embodiments, the mean sphere and/or the cylinder increase function along the at least one section is a Quadratic function.

According to some embodiments, the optical elements are configured so that the mean focus of the light rays passing through each optical element is at a same distance to the retina.

According to some embodiments, the refractive area is formed as the area other than the areas formed as the plurality of optical elements.

According to some embodiments, for every circular zone having a radius comprised between 2 and 4 mm comprising a geometrical center located at a distance of the optical center of the lens greater or equal to said radius+5 mm, the ratio between the sum of areas of the parts of optical elements located inside said circular zone and the area of said circular zone is greater than or equal to 20%, preferably greater than or equal to 30%, more preferably greater than or equal 40% and smaller than or equal to 70%, preferably smaller than or equal to 60%, more preferably smaller than or equal to 50%.

According to some embodiments, at least one of the optical elements is a multifocal refractive lenslet.

According to some embodiments, the at least one multifocal refractive lenslet comprises a cylindrical power.

According to some embodiments, the at least one multifocal refractive lenslet comprises an aspherical surface, with or without any rotational symmetry.

According to some embodiments, the at least one multifocal refractive lenslet comprises a spherical surface.

According to some embodiments, at least one of the optical elements is a toric refractive lenslet.

According to some embodiments, the at least one multifocal refractive lenslet comprises a toric surface.

According to some embodiments, at least one of the optical elements is made of a birefringent material.

According to some embodiments, at least one of the optical elements is a diffractive lens.

According to some embodiments, the at least one diffractive lens comprises a metasurface structure.

According to some embodiments, at least one optical element has a shape configured so as to create a caustic in front of the retina of the eye of the person. In other words, such optical element is configured so that every section plane where the light flux is concentrated if any, is located in front of the retina of the eye of the person.

According to some embodiments, at least one optical element is a multifocal binary component.

According to some embodiments, at least one optical element is a pixelated lens.

According to some embodiments, at least one optical element is a n-Fresnel lens.

According to some embodiments, at least one, for example all, optical elements comprise high order optical aberrations.

According to some embodiments, the lens comprises an ophthalmic lens bearing the refractive area and a clip-on bearing the optical elements adapted to be removably attached to the ophthalmic lens when the lens is worn.

According to some embodiments, the refractive area is further configured to provide to the wearer and for foveal vision a second refractive power different from the first refractive power.

According to some embodiments, the difference between the first refractive power and the second refractive power is greater than or equal to 0.5 diopter.

According to some embodiments, at least one, for example at least 70%, for example all optical elements are active optical element that may be activated by an optical lens controller.

According to some embodiments, the active optical element comprises a material having a variable refractive index whose value is controlled by the optical lens controller.

According to some embodiments, the optical elements are not visible on the lens.

According to another aspect, the invention relates to a computer-implemented method for determining a lens intended to be worn by a wearer, to provide a refractive power based on a prescription of the wearer for correcting an abnormal refraction of the eye of the wearer.

According to some embodiments, the lens is a spectacle lens intended to be worn in front of an eye of a wearer. According to some embodiments, the lens is a contact lens intended to be worn on an eye of a wearer.

According to some embodiments, the lens is intended to provide a refractive power (Rx) based on a prescription of the wearer for correcting an abnormal refraction of the eye of the wearer in standard wearing conditions.

According to some embodiments, the lens is intended to suppress or slow down the progression of an abnormal refraction of said eye of the wearer.

The lens comprises a refractive area configured to provide the refractive power (Rx) and a functional area configured to provide an optical function.

According to some embodiments, the lens consists of the refractive area and the functional area.

According to some embodiments, the functional area is not configured to provide the first refractive power (Rx). According to some embodiments, the functional area is not configured to provide only the first refractive power (Rx). It means that the functional area may be configured to provide the first refractive power (Rx), or a second refractive power different from the first refractive power, together with an additional optical function. According to some embodiments, the functional area is configured to provide an optical function different from the first refractive power (Rx).

According to some embodiments, the functional area is defined has any area having a local refractive power above the first refractive power (Rx) plus 1 diopter or below the first refractive power (Rx) minus 1 diopter. According to some embodiments, the functional area is defined has any area wherein the difference between the local refractive power and the first refractive power (Rx) is above 1 diopter in absolute value.

According to some embodiments, the functional area is configured to suppress or slow down the progression of an abnormal refraction of said eye of the wearer.

According to some embodiments, the functional area has an optical function of not focusing an image on the retina of the eye of the wearer, an optical function of scattering incident light or an optical function of focusing an image other than on the retina of the eye of the wearer.

According to some embodiments, the functional area comprises an optical microstructure. According to some embodiments, said optical microstructure has an optical function of not focusing an image on the retina of the eye of the wearer so as to slow down the progression of the abnormal refraction of the eye of the wearer.

According to some embodiments, the functional area comprises an optical microstructure having an optical function of not focusing an image on the retina of the eye of the wearer in standard wearing conditions so as to slow down the progression of the abnormal refraction of the eye of the wearer.

The method comprises the following steps:
providing a prescription of the wearer prescribing a first refractive power (Rx);
determining the refractive area so that on a first zone, for example having a circular shape, defined on the lens, more than 5%, for example more than 7% and for example less than 39%, of the surface of said first zone has a local refractive power ranging from the first refractive power (Rx) minus 0.25 diopter and the first refractive power (Rx) plus 0.25 diopter, and more than 25%, for example more than 30%, and for example less than 60%, of the surface of said first zone covers the functional area.

According to some embodiments, the method further comprises the step of providing a wearing condition of the lens by the wearer.

According to some embodiments, the method for determining the refractive area comprises:
a first step of defining initial parameters of the functional area;
a second step of determining parameters of the lens to provide the prescription of the wearer; and
a third step of optimizing the initial parameters so that on a first zone defined on the lens, more than 5%, for example more than 7%, of the surface of said first zone has a local refractive power ranging from the first refractive power (Rx) minus 0.25 diopter and the first refractive power (Rx) plus 0.25 diopter, and more than 25%, for example more than 30%, of the surface of said first zone covers the functional area.

According to some embodiments, during the first step initial parameters of the functional area are defined. Such initial parameters can be selected from
the size of the optical microstructure, for instance the size of the optical elements or the distance between optical elements,
the optical properties of the optical microstructure, such as for instance spherical, non-spherical, spherico-cylindrical, aspherical, refractive, or diffractive optical elements,
the ratio between the surface of the functional area and the surface of the refractive area,
the position of the microstructure, such as the position of the optical elements in a network. The network may be a random network or a structured network, such as for instance a squared network, a hexagonal network, a triangle network, an octagonal network, a honeycomb, or concentric rings,
the position of the microstructure on an object-side surface and/or an eye-side surface and/or between the object-side surface and the eye-side surface of the lens.

According to some embodiments, during the second step the material of the lens with a known refractive index, the front surface and the back surface are determined so that the lens provides the prescription of the wearer.

According to some embodiments, the front surface is spherical, aspherical, or progressive.

According to some embodiments, the back surface is spherical cylindrical, aspherical, atorical or progressive.

According to some embodiments, during the third step the surface of the first zone having a local refractive power ranging from the first refractive power (Rx) minus 0.25 diopter and the first refractive power (Rx) plus 0.25 diopter is computed as mentioned above. If the surface of the first zone having a local refractive power ranging from the first refractive power (Rx) minus 0.25 diopter and the first refractive power (Rx) plus 0.25 diopter is not higher than 5%, for example not higher than 7%, the initial parameters are iteratively modified until the surface satisfies the threshold.

According to some embodiments, the optimization procedure is a constrained optimization procedure so that the optical microstructure further respects one of the following criteria.

According to some embodiments, the method further comprises the step of determining the refractive area so that more than 10% of the surface of said first zone has a local refractive power ranging from the first refractive power (Rx) minus 0.25 diopter and the first refractive power (Rx) plus 0.25 diopter; or more than 15% of the surface of said first zone has a local refractive power ranging from the first refractive power (Rx) minus 0.25 diopter and the first refractive power (Rx) plus 0.25 diopter; or more than 20% of the surface of said first zone has a local refractive power ranging from the first refractive power (Rx) minus 0.25 diopter and the first refractive power (Rx) plus 0.25 diopter; or more than 25% of the surface of said first zone has a local refractive power ranging from the first refractive power (Rx) minus 0.25 diopter and the first refractive power (Rx) plus 0.25 diopter.

According to some embodiments, the method further comprises the step of determining the refractive area so that more than 15% of the surface of said first zone has a local refractive power ranging from the first refractive power (Rx) minus 0.5 diopter and the first refractive power (Rx) plus 0.5 diopter; or more than 20% of the surface of said first zone has a local refractive power ranging from the first refractive power (Rx) minus 0.5 diopter and the first refractive power (Rx) plus 0.5 diopter; or more than 25% of the surface of said first zone has a local refractive power ranging from the first refractive power (Rx) minus 0.5 diopter and the first refractive power (Rx) plus 0.5 diopter; or more than 30% of the surface of said first zone has a local refractive power ranging from the first refractive power (Rx) minus 0.5 diopter and the first refractive power (Rx) plus 0.5 diopter; or more than 35% of the surface of said first zone has a local refractive power ranging from the first refractive power (Rx) minus 0.5 diopter and the first refractive power (Rx) plus 0.5 diopter.

According to some embodiments, the method further comprises the step of determining the refractive area so that less than 39% of the surface of said first zone has a local refractive power superior or equal to the first refractive power (Rx) plus 0.12 diopter; or less than 37% of the surface of said first zone has a local refractive power superior or equal to the first refractive power (Rx) plus 0.12 diopter; or less than 30% of the surface of said first zone has a local refractive power superior or equal to the first refractive power (Rx) plus 0.12 diopter.

According to some embodiments, the method further comprises the step of determining the refractive area so that less than 70% of the surface of the first zone has a local refractive power inferior or equal to the first refractive power (Rx) minus 0.12 diopter; or less than 68% of the surface of the first zone has a local refractive power inferior or equal to the first refractive power (Rx) minus 0.12 diopter; or less than 65% of the surface of the first zone has a local refractive power inferior or equal to the first refractive power (Rx) minus 0.12 diopter; or less than 62% of the surface of the first zone has a local refractive power inferior or equal to the first refractive power (Rx) minus 0.12 diopter; or less than 60% of the surface of the first zone has a local refractive power inferior or equal to the first refractive power (Rx) minus 0.12 diopter; or less than 55% of the surface of the first zone has a local refractive power inferior or equal to the first refractive power (Rx) minus 0.12 diopter; or less than 50% of the surface of the first zone has a local refractive power inferior or equal to the first refractive power (Rx) minus 0.12 diopter.

According to some embodiments, the method further comprises the step of determining the refractive area so that more than 3% of the surface of the first zone has a local refractive power ranging from the first refractive power (Rx) plus 0.12 diopter and the first refractive power (Rx) minus 0.12 diopter; or more than 4% of the surface of the first zone has a local refractive power ranging from the first refractive power (Rx) plus 0.12 diopter and the first refractive power (Rx) minus 0.12 diopter; or more than 4.5% of the surface of the first zone has a local refractive power ranging from the first refractive power (Rx) plus 0.12 diopter and the first refractive power (Rx) minus 0.12 diopter; or more than 5% of the surface of the first zone has a local refractive power ranging from the first refractive power (Rx) plus 0.12 diopter and the first refractive power (Rx) minus 0.12 diopter; or more than 10% of the surface of the first zone has a local refractive power ranging from the first refractive power (Rx) plus 0.12 diopter and the first refractive power (Rx) minus 0.12 diopter; or more than 13% of the surface of the first zone has a local refractive power ranging from the first refractive power (Rx) plus 0.12 diopter and the first refractive power (Rx) minus 0.12 diopter; or more than 14% of the surface of the first zone has a local refractive power ranging from the first refractive power (Rx) plus 0.12 diopter and the first refractive power (Rx) minus 0.12 diopter; or more than 15% of the surface of the first zone has a local refractive power ranging from the first refractive power (Rx) plus 0.12 diopter and the first refractive power (Rx) minus 0.12 diopter.

In the description, terms like «up», «bottom», «horizontal», «vertical», «above», «below», «front», «rear» or other words indicating relative position may be used. These terms are to be understood in the wearing conditions of the lens.

The term “horizontal” refers here to the horizontal with respect to the wearer according to the TABO convention. Likewise, the term “vertical” refers here to the vertical with respect to the wearer according to the TABO convention.

In the context of the present invention, the term “lens” can refer to an uncut optical lens or a spectacle optical lens edged to fit a specific spectacle frame or an ophthalmic lens.

While various embodiments have been described and illustrated, the details description and drawings should not be considered as restrictive but merely exemplary and illustrative. Various modifications can be made to the embodiments by those skilled in the art without departing from the scope of the disclosure as defined by the claims. In the claims, the word “comprising” does not exclude other elements or steps, and the indefinite article “a” or “an” does not exclude a plurality. The mere fact that different features are recited in mutually different dependent claims does not indicate that a combination of these features cannot be advantageously used. Any reference signs in the claims should not be construed as limiting the scope of the invention.

1. A spectacle lens intended to be worn in front of an eye of a wearer and to provide a first refractive power for correcting an abnormal refraction of the eye of the wearer, the lens comprising:
a refractive area configured to provide the first refractive power and on at least one surface a functional area configured to provide an optical function; and
a coating covering the at least one surface having the functional area,
wherein on a first zone having a circular shape defined on the lens, more than 5% and less than 39% of the surface of said first zone has a local refractive power ranging from the first refractive power minus 0.25 diopter and the first refractive power plus 0.25 diopter, and
wherein more than 25% and less than 60% of the surface of said first zone covers the functional area.

2. The lens according to claim 1, wherein more than 10% of the surface of said first zone has a local refractive power ranging from the first refractive power minus 0.25 diopter and the first refractive power plus 0.25 diopter.

3. The lens according to claim 1, wherein more than 15% of the surface of said first zone has a local refractive power ranging from the first refractive power minus 0.5 diopter and the first refractive power plus 0.5 diopter.

4. The lens according to claim 3, wherein more than 20% of the surface of said first zone has a local refractive power ranging from the first refractive power minus 0.5 diopter and the first refractive power plus 0.5 diopter, or
wherein more than 25% of the surface of said first zone has a local refractive power ranging from the first refractive power minus 0.5 diopter and the first refractive power plus 0.5 diopter, or
wherein more than 30% of the surface of said first zone has a local refractive power ranging from the first refractive power minus 0.5 diopter and the first refractive power plus 0.5 diopter; or wherein more than 35% of the surface of said first zone has a local refractive power ranging from the first refractive power minus 0.5 diopter and the first refractive power plus 0.5 diopter.

5. The lens according to claim 1, wherein less than 39% of the surface of said first zone has a local refractive power superior or equal to the first refractive power plus 0.12 diopter; or wherein less than 37% of the surface of said first zone has a local refractive power superior or equal to the first refractive power plus 0.12 diopter, or
wherein less than 30% of the surface of said first zone has a local refractive power superior or equal to the first refractive power plus 0.12 diopter.

6. The lens according to claim 1, wherein the first zone has a diameter greater than or equal to 6 mm and smaller than or equal to 10 mm.

7. The lens according to claim 1, wherein the functional area has an optical function of not focusing an image on a retina of the eye of the wearer or an optical function of scattering incident light.

8. The lens according to claim 1, wherein the functional area includes an optical microstructure.

9. The lens according to claim 8, wherein the optical microstructure comprises includes a plurality of optical elements, such as for instance lenslets.

10. The lens according to claim 8, wherein the optical microstructure is disposed on an object-side surface and/or an eye-side surface and/or between the object-side surface and the eye-side surface of the lens.

11. The lens according to claim 9, wherein the plurality of optical elements is positioned randomly or in a network, including a regular network being a grid, a honeycomb, or concentric rings.

12. The lens according to claim 9, wherein the smallest distance between two adjacent and non-contiguous optical elements is greater than or equal to 0.2 mm or 0.3 mm, and smaller than or equal to 2 mm or 1.5 mm.

13. The lens according to claim 1, wherein the lens includes a coating on a surface including the functional area.

14. The lens according to claim 1, wherein the refractive area and/or the functional area is a connected area or a non-connected area.

15. A computer-implemented method for determining a spectacle lens intended to be worn in front of an eye of a wearer, to provide a first refractive power based on a prescription of the wearer for correcting an abnormal refraction of the eye of the wearer, wherein the lens includes a refractive area configured to provide the first refractive power and on at least one surface a functional area configured to provide an optical function and a coating covering the at least one surface having the functional area the method comprising:
obtaining a prescription of the wearer; and
determining the refractive area so that on a first zone having a circular shape defined on the lens, more than 5% and less than 39% of the surface of said first zone has a local refractive power ranging from the first refractive power minus 0.25 diopter and the first refractive power plus 0.25 diopter, and more than 25% and less than 60% of the surface of said first zone covers the functional area.

16. The lens according to claim 2, wherein more than 15% of the surface of said first zone has a local refractive power ranging from the first refractive power minus 0.5 diopter and the first refractive power plus 0.5 diopter.

17. The lens according to claim 9, wherein the optical microstructure is disposed on an object-side surface and/or an eye-side surface and/or between the object-side surface and the eye-side surface of the lens.

18. The lens according to claim 17, wherein the plurality of optical elements is positioned randomly.

19. The lens according to claim 17, wherein the plurality of optical elements is positioned in a network.

20. The lens according to claim 17, wherein the plurality of optical elements is positioned in a regular network including a grid, a honeycomb, or concentric rings.
